(19) United States
(12) Patent Application Publication
(10) Pub. No.: US 2022/0203113 A1
(43) Pub. Date: Jun. 30, 2022

(54) MAGNETIC NANOPARTICLE PLAQUE CLEARANCE

(71) Applicant: Trevor Percival Castor, Arlington, MA (US)

(72) Inventor: Trevor Percival Castor, Arlington, MA (US)

(73) Assignee: Trevor P. Castor, Woburn, MA (US)

(21) Appl. No.: 17/561,799

(22) Filed: Dec. 24, 2021

Related U.S. Application Data
(60) Provisional application No. 63/130,524, filed on Dec. 24, 2020.

(51) Int. Cl. A61N 2/00; A61N 2/02

(52) U.S. Cl. CPC A61N 2/004; B82Y 25/00; A61N 2/02

(57) ABSTRACT

This invention relates to methods and systems for removing plaque from arteries and blood vessels in human subjects non-invasively utilizing coated superparamagnetic nanoparticles introduced into the human bloodstream and controlled by external magnetic fields to effect plaque removal. Magnetic nanoparticles are injected into a patient, and magnetic fields are used to move the nanoparticles to the site of an arterial blockage. The nanoparticles are then oscillated by means of an alternating current source and oscillating magnetic field. The nanoparticles impact the plaque deposit, causing it to break up, so that it may be safely disintegrated, dissolved in the bloodstream, digested by enzymes or ions and/or removed with the nanoparticles themselves. The nanoparticles are removed by a unipolar magnetic field directing them to be removed at the point of injection or alternative location in the body. The nanoparticles are also removed by natural bodily functions. The methods and systems are directed to the emergency clearance of arteries in the human body, and the routine annual and quarterly clearance and preventative maintenance of arteries in the human body.

### GOVERNMENT SUPPORT

Embodiments of the present invention were conceived and reduced to practice without Federal sponsorship or funding.

### FIELD OF THE INVENTION

This invention relates to methods and systems for removing plaque from arteries and blood vessels in human subjects non-invasively utilizing coated superparamagnetic nanoparticles introduced into the human bloodstream and controlled by external magnetic fields to effect plaque removal.

### BACKGROUND OF THE INVENTION

Atherosclerosis is a major health risk. In this progressive condition, also called coronary artery disease, fatty material, consisting of fat, cholesterol and other substances, collects on artery walls over time. As this fatty material hardens, it forms calcium deposits called plaques. The build-up of plaque makes a blood vessel narrow and less flexible and eventually, may block blood flow. Reduced blood flow in the coronary arteries may cause chest pain called angina, shortness of breath, a heart attack and other symptoms. Atherosclerosis often has no symptoms until a plaque ruptures or the buildup is severe enough to obstruct blood flow.

In an unstable condition called atherosclerotic plaque, small pieces of plaque may break away and lodge in smaller blood vessels, blocking blood flow. This blockage, called an embolization, is a common cause of heart attack and stroke. Blood clots can also form around a tear in the plaque leading to a blockage. A blood clot that moves into an artery in the heart, lungs, or brain can cause a stroke, heart attack, or pulmonary embolism. Plaque may also weaken the wall of an artery leading to an aneurysm.

A buildup of cholesterol plaques in the walls of arteries may cause obstruction of blood flow. Plaques may rupture causing acute occlusion of the artery by clot.

Although some medications and medical procedures are being developed to open blocked arteries, with surgery as a more invasive solution, most physicians recommend a healthy diet and exercise to control the build-up of arterial plaques.

There are several known methods for detecting plaque deposits in arteries. Some of these methods are highly invasive, and others are less so. Today, many patients are evaluated for coronary artery disease by invasive tests such as angiography, which involves inserting a catheter into the body and threading it into the aorta. Molecular and nuclear imaging offer the opportunity to assess blood flow non-invasively, without making a surgical incision or inserting a medical instrument into the body.

Heart scans, known as coronary calcium scans, are specialized X-ray tests that provide images to locate and provide measurements of calcium-containing plaque in the arteries. Plaque deposits can grow over time and restrict blood flow to the heart. Another method of detecting arterial plaque is an ultrasonic scan, which can provide an image of plaque location and size.

Still another method detecting and locating arterial plaque includes injecting a dye into a patient which contains a radioactive isotope such as technetium 99m, which has a short radioactive half-life. The radioactive dye is absorbed by the plaque deposit so that it may be detected noninvasively by a scanner.

Although these and other methods are known for detecting plaque, there are few solutions for removing plaque in a safe manner, without invasive techniques such as surgery. Surgery carries inherent risks, since soft plaque deposits can rupture and cause a heart attack or stroke.

There is a pressing need for a method of removing plaque from arteries and the related vasculature in a safe, noninvasive manner.

### SUMMARY OF THE INVENTION

Research is being conducted using drug-loaded magnetic microspheres for the targeted delivery of anticancer drugs in a process known as targeted chemotherapy. Using an externally applied magnetic field, the drug-loaded nanoparticles may be manipulated to the location of a tumor through the human vascular system, where the chemotherapy drugs may be released in a specific target area. In other research, magnetic nanoparticles are manipulated in the blood stream to the location of the tumor, and the nanoparticles are heated by magnetic induction to a sufficient temperature to kill cancer cells in the proximity of the heated nanoparticles.

The present invention is related to nanoparticles but it addresses a different problem. The problem is to cleanse plaque deposits from the arteries of a human subject without using intrusive methods. The inventor's solution is a system and method for introducing a plurality of polymer-coated superparamagnetic nanoparticles into the bloodstream in the vicinity of a plaque deposit, and vibrate the nanoparticles in proximity of a plaque deposit to physically abrade and dislodge the plaque deposit, and allow the plaque deposit to harmlessly dissolve in the bloodstream.

Performed throughout the vascular system of the human subject systematically, the magnetic plaque clearance system and method of the present invention will provide a seemingly rejuvenating effect to a patient's circulatory system by removing plaque deposits which could later cause artery blockages.

A solution containing a plurality of superparamagnetic polymer-coated nanoparticles are introduced into the human subject's bloodstream by means of a syringe or any other convenient method. The nanoparticles are dispersed, and using an externally applied electromagnetic field, the nanoparticles are progressively moved through the subject's vascular system to the specific location of a plaque deposit. The density of the nanoparticles at a particular location is maximized by the application of an external electromagnetic field.

The electromagnetic field is then changed from a direct one to an oscillating one in order to disrupt or disintegrate the plaques. Disintegrated particles that do not directly dissolve into the blood stream are digested by enzymes on the coating of the nanoparticle or by enzymes introduced into the environment of the plaque. Additionally, or alternatively, disintegrated particles such as calcium deposits that do not directly dissolve into the blood stream are dissolved by hydroxyl or electron donating anions on the coating of the nanoparticle or by hydroxyl or electron donating anions introduced into the environment of the plaque.

The electromagnetic field is then changed from an oscillation one to a direct one in order to move the nanoparticles to a point of removal from the body.

The nanoparticles are removed by a unipolar magnetic field directing them to be removed at the point of injection or alternative location in the body. The nanoparticles are also removed by natural bodily functions.

The methods and systems are directed to the emergency clearance of arteries in the human body, and the routine annual and quarterly clearance and preventative maintenance of arteries in the human body.

The envisioned invention describes a process for the emergency clearance of arteries in the human body and the routine periodic annual and quarterly clearance and preventative maintenance of arteries in the human body.

### BRIEF DESCRIPTION OF THE FIGURES

FIG. 1 shows a schematic diagram showing the cross-section of an artery in which a plaque deposit grows over time to finally cause a nearly complete blockage of the artery;

FIG. 2 illustrates an artery with severe plaque deposits which cause obstruction of the flow of red blood cells in the vicinity of the obstruction;

FIG. 3 is a schematic diagram showing the basic operating principle of the present invention particularly how magnetic nanoparticles are controlled by a magnetic field;

FIG. 4 shows the structure of a magnetic nanoparticle or nanosphere in accordance with one embodiment of the present invention wherein the nanoparticles include a polymer coating and additionally an enzyme coating and/or anionic coating;

FIG. 5 illustrates the arrangement of elements for generating an external electromagnetic field to control the location and activity of magnetic nanoparticles in the vicinity of an artery blockage caused by plaque; and

FIG. 6 shows a flowchart of the process flow for removing a plaque deposit from an artery.

### DETAILED DESCRIPTION

It is intended that the matter contained in the preceding description be interpreted in an illustrative rather than a limiting sense.

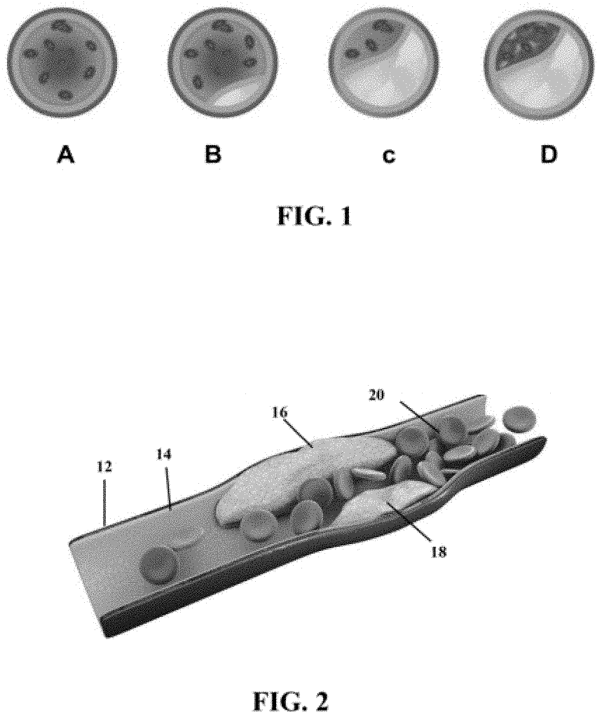

Turning to FIG. 1, an artery is shown schematically in cross-section in the process of a degenerative blockage forming over time. In FIG. 1A, normal artery is shown which is completely unobstructed and free of arterial plaque. FIG. 1B in the lower portion of the artery and the beginning of plaque accumulation in the artery. This is the initial stage of arterial-sclerosis. Cholesterol is collecting in the artery and decreasing blood flow. In FIG. 1C, the artery is shown with advanced cholesterol plaque blocking the major portion of the artery. This shows the instance of significant a theory atherosclerosis forming. An extremely serious health risk is presented to the patient at this point in this continuing degenerative process. Finally, in FIG. 1D, the blockage is almost complete and blood flow is almost fully restricted. This is the last stage and possibly the final stage of atherosclerosis, with the patient's life is in extreme jeopardy.

FIG. 2 diagrammatically illustrates the final stage of atherosclerosis. The artery 12 has an exterior wall 12 and an interior wall 14, and blood flows on the inside of the interior wall 14. In this diagram, two huge plaque deposits are shown (16,18) which have attached themselves to the interior artery wall 14 and have expanded almost to the level where blood flow is fully impeded. Red blood cells 20 are shown in the diagram as being blocked from passing freely around the plaque deposits which have accumulated in the artery. The plaque (16,18) consists of cholesterol and fatty materials that have deposited over time, blocking the flow of blood.

### Overview

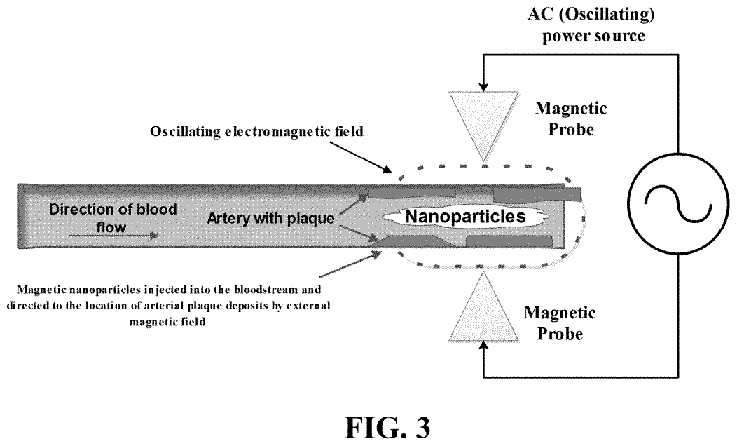

FIG. 3 shows, diagrammatically, the operation of the present invention. An aqueous solution containing the plurality of superparamagnetic nanoparticles, which will be described further on, is injected into the patient. The location of the injection is chosen with respect to the location of the arterial plaque deposit, which is determined by one of several methods, as will be described.

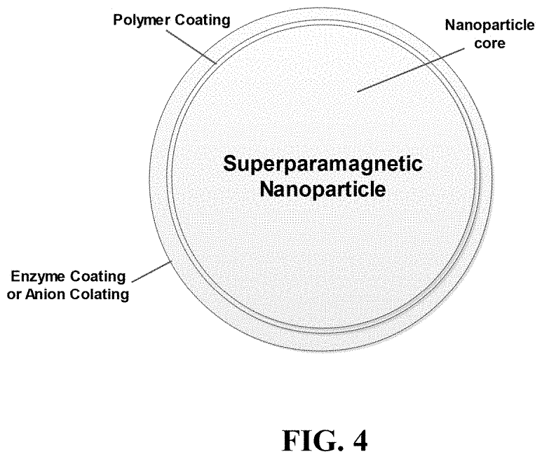

It is known how to generate strong electromagnetic fields which can be focused to a specific point. The present system generates a focused electromagnetic field, much as a solenoid produces a focused electromagnetic field. Additional concepts to increase magnetic field strength at a point location include the use of cone magnets or pyramid magnets to focus and intensify an electromagnetic field to a particular point location. The tapered conical shape produces a concentrated and more intense magnetic field on the tapered tip, compared to the base part of the magnetic core. Using this arrangement, and electromagnetic field can be focused and intensified at a particular point location. This will be discussed further on in connection with FIG. 4.

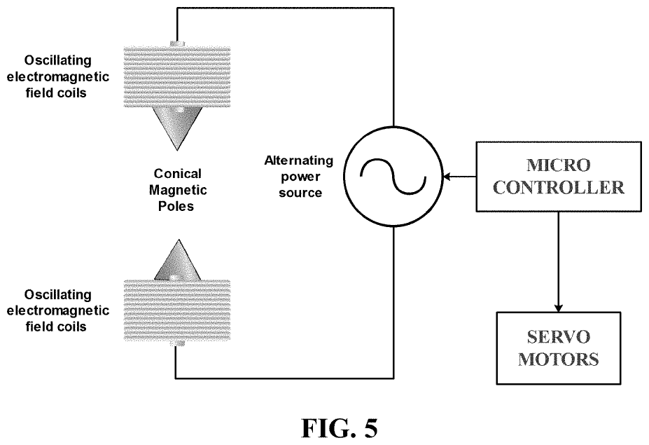

The plurality of magnetic nanoparticles flows along with the blood flow in the artery, and the nanoparticles are attracted to the location of the plaque deposits in the artery. The magnetic field is operating in steady-state, so that the magnetic nanoparticles may be directed to the desired location in close proximity to the plaque deposit, at the location of the magnetic probes shown in FIG. 5.

At this point in the process, the magnetic field under the control of a microcontroller, switches to an oscillating power source. An oscillating electromagnetic field is now generated in the vicinity of the plaque deposits. The nanoparticles vibrate or oscillate in response to the alternating current power source. There is an agitation of the magnetic nanoparticles, in a manner wherein the nanoparticles abrade the plaque deposit. The nanoparticles impact the surface of the plaque deposit and break it up and eventually destroy it. The plaque is reabsorbed into the blood stream to be carried away harmlessly or destroyed by coatings on the magnetic nanoparticles as will be described.

At the conclusion of the treatment, the nanoparticles will be attracted to a location within the patient, and since they are concentrated in one area, they may be conveniently removed from the body in a magnetically biased syringe or other means to destroy them.

### Magnetic Nanoparticles

Turning now to FIG. 4, a superparamagnetic nanoparticle is shown diagrammatically. Magnetic nanoparticles (MNPs) are being used as image contrast probes, hydrothermal agents, magnetic-guide vectors and drug delivery carriers. The main advantages of using MNPs for such purposes include easy preparation, small sizes (>30 nm), chemical functionalization, biocompatibilities and stabilities, efficient drug conjugation and superior magnetic responsiveness.

The most widely used systems in biological settings are MNPs made of iron oxides (Fe₃O₄/Fe₂O₃) due to their well-known biocompatibilities (Longmire et al., 2008). When the size of the MPN is below a critical value (˜30 nm), these nanoparticles behave like a giant paramagnetic atom with a single magnetic domain exhibiting superparamagnetic behavior. Superparamagnetic nanoparticles respond rapidly to an applied magnetic field with negligible residual magnetism away from the magnetic field and when the magnetic field is turned off or removed.

In the present invention, the superparamagnetic nanoparticles are preferably spherical in shape. A magnetic core, manufactured from a ferrite material, is surrounded by a polymer-coat. In another embodiment, additional coatings of enzymes and and/or acidic moieties are applied to the nanoparticles for the purpose of dissolving or destroying plaque particles released during the breakup of the plaque deposit. This is for the purpose of preventing dislodged plaque particles from traveling upstream of the blockage and causing additional blockages in smaller blood vessels.

### Generating the Magnetic Field

FIG. 5 shows in more detail the basic circuitry used for generating an oscillating electromagnetic field, as described earlier on. The electromagnet probes consist of a densely coiled magnetic core with conical magnetic poles. The core generates an intense magnetic field and the conical magnetic poles focus that magnetic field on a chosen location in proximity to the plaque deposit or blockage.

An electromagnetic field generator using known techniques can generate sufficient magnetic field strength to effectively influence the movement of superparamagnetic nanospheres circulating in the vascular system of a patient. The magnetic field generator has two modes of operation. In the steady-state mode, the nanoparticles are moved as a group linearly to the location of the plaque deposit. In the second operational mode, the power source is alternated to generate an oscillating electromagnetic field, imparting vibrational or oscillatory motion to the nanoparticles so that the nanoparticles can be directed to abrade the plaque deposit and break up or dislodge the plaque deposit.

The externally-generated electromagnetic field is focused on a specific location, such as a small area in an artery, where it is previously determined (by MRI or other detection) that plaque buildup is present. The focused magnetic field attracts the polymer coated superparamagnetic nanospheres to that particular location in an artery.

The external electromagnetic field is mobile, so that it can be moved progressively to various locations in the patient's body. In this way, the focus magnetic field (1) attracts the nanoparticles to particular location adjacent to the location of a plaque deposit; and (2) imparts and oscillatory motion to the nanoparticles in the vicinity of the plaque deposit. This oscillatory motion will cause the plurality of nanoparticles to interact by abrasion with the plaque deposit. The polymer coating on the nanoparticles will minimize any damaging effect to exposed epithelial tissue in the artery walls from the vibrational impact of the nanoparticles or nanospheres.

In use, the conical magnetic poles of the electromagnet system, are positioned to be on opposite sides of the obstructed artery, so that a proper oscillatory motion can be imparted to the superparamagnetic nanoparticles, so that they will oscillate in a predictable manner, as determined by the microcontroller system.

### Programmed Microcontroller

In FIG. 5, an electronic controller will determine whether the magnetic field is static (for attracting the nanoparticles to particular location in the artery); or oscillatory (for causing vibrational movement of the magnetic nanoparticles, in the artery). The focused electromagnetic field uses a magnetic pole reversal system through use of an oscillator to impart the vibratory motion to the nanoparticles in the vicinity of a plaque deposit. The electronic controller will further control several magnetic parameters including electromagnetic field strength, operational duration of the electromagnetic field, nanoparticles and vibration rate, nanoparticles vibration magnitude. In some embodiments, the rotation of the nanoparticles will be controlled by the microcontroller controlling the electromagnetic field.

The external electromagnets will move incrementally and progressively along all the major arteries of the human subject's vasculature, removing plaque deposits encountered along the way. At the completion of the treatment, the magnetic nanoparticles will be recalled (redirected) to the entry or alternative site, so that they can be removed via removal methods such as a syringe.

In another embodiment, the magnetic probes may be located on a mechanical frame with movement controlled by multiple servo mechanisms, under the control of a programmable microcontroller. In this embodiment, the magnetic probes will be moved to a programmed position relative to the patient's body so that the concentrated magnetic field is applied to a specific location corresponding to the location of the plaque deposit, which has been detected by other means.

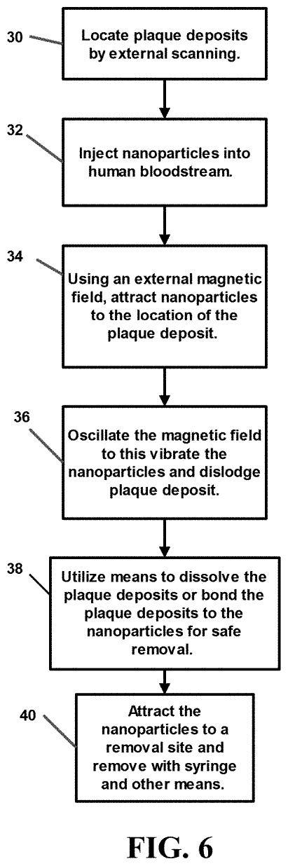

Turning to FIG. 6, a block diagram is shown whereof one embodiment of the entire process is for plaque removal from arteries. In the step 30, the location of plaque deposits is determined by external scanning. Plaque deposits may be located by any of several known methods including radioactive dyes using short-lived radioisotopes such as technetium 99m, ultrasound, MRI or PET scanning, angiogram, and CT scans. Once the location of an arterial plaque deposit is identified by diagnostic means, the programmable controller will move the mechanical frame and magnetic probes to the location of the plaque deposit.

In the next step 32 of FIG. 6, the superparamagnetic nanoparticles, preferably in the shape of nanospheres, will be injected in an aqueous solution into the blood stream at a predetermined location. In the first mode of operation, the magnetic probes will guide the magnetic nanospheres, step 34, through the bloodstream to the location of the arterial plaque deposit.

In the second mode of operation, step 36, a microcontroller switches the magnetic field to an alternating current mode, which will be an oscillating magnetic field directed to the location of the arterial plaque. The microcontroller will control the time duration of the treatment procedure, in which the nanospheres will be vibrated or oscillated by the oscillating magnetic field in the vicinity of the plaque deposit. The oscillating action of the nanospheres will have an abrading effect on the plaque deposit breaking it up and dislodging it into small particles which will be readily absorbed into the bloodstream. The treatment will be under control of the microcontroller, which will control the duration of the treatment and the field strength of the magnetic probes, so that the oscillating action of the magnetic will be adjustable to provide optimum treatment of the particular deposit of plaque on the artery wall.

In another embodiment, the microcontroller will receive feedback data from an imaging system to monitor the progression of the treatment process. In a real-time, the program microcontroller monitors the process and determines the effect of the nanospheres on the plaque deposit during the treatment process, so that field strength, oscillation rate, and treatment duration can be adjusted in real time.

### Enzyme Coating of Nanoparticles and Removal of Nanospheres

As plaque deposits break up into small particles, there is a possibility that the smaller particles could in themselves cause blockages in smaller blood vessels. In step 38 of FIG. 6, the nanoparticles themselves can prevent this situation by providing them with an exterior coating in the form of an enzyme which will digest the particles as they break away from the plaque deposit. The enzymes can also be introduced into the bloodstream and environment of the plaque through an alternative source.

The enzymes, in this embodiment are designed to absorb or digest by using biochemical action to remove the dissolved plaque particles from the bloodstream so that they do not deposit downstream in other blood vessels.

Additionally, or alternatively, disintegrated particles such as calcium deposits that do not directly dissolve into the blood stream are dissolved by hydroxyl or electron donating anions on the coating of the nanoparticle or by hydroxyl or electron donating anions introduced into the environment of the plaque.

Alternatively, the nanoparticles could be charged by the proper anions or the like, so that the dislodged particles could bond to the magnetic nanoparticles and be safely removed at the same time the nanoparticles are removed at the removal site by syringe or other means in step 40 once the nanoparticles are attracted to the removal site.

While this invention has been particularly shown and described with references to specific embodiments, it will be understood by those skilled in the art that various changes in form and details may be made therein without departing from the spirit and scope of the invention as defined by the appended claims.

What is claimed are:

1. A method of treating a human subject for removing plaque deposits from blood vessels and vasculature of the human subject, the method comprising:
introducing a plurality of superparamagnetic nanoparticles into the bloodstream of a human subject;
generating a rapidly changing, time-varying electromagnetic field external to the human subject, the electromagnetic field having sufficient magnetic field strength to encompass the human subject, wherein the plurality of superparamagnetic nanoparticles is distributed substantially uniformly though the vasculature of a human subject;
controlling the time-varying electromagnetic field generating an oscillating magnetic effect magnetic to impart randomized vibrational motion to the superparamagnetic nanoparticles throughout the bloodstream of the human subject, wherein the agitating motion of the superparamagnetic nanoparticles vibrate while passing through the vasculature of the human subject, so as to substantially dislodge and dissolve accumulated plaque in the individual blood vessels; and
using a unipolar magnetic field, removing the superparamagnetic nanoparticles from the bloodstream of the human subject.

2. The method of claim 1, further including programming a controller to cause the electromagnetic field to operate for a predetermined duration of treatment time.

3. The method of claim 1, wherein the electromagnetic field is focused on a designated sub portion of the vasculature of the human subject, to provide localized treatment of designated blood vessels and vasculature.

4. The method of claim 1, wherein the field strength of the electromagnetic field is controllable for designated human subject.

5. The method of claim 1, wherein the unipolar magnetic field is provided by a separate treatment station from the electromagnetic field.

6. The method of claim 1, wherein the nanoparticle removal takes place after a predetermined time duration.

7. The method of claim 1 wherein the nanoparticle removal occurs immediately following the treatment.

8. The method of claim 1, wherein the time-varying electromagnetic field is created by an electromagnetic field generator.

9. The method of claim 1, wherein the superparamagnetic nanoparticles removal occurs during a post-treatment dialytic procedure.

10. The method of claim 1, wherein the oscillating magnetic effect is focused on a predetermined subset of blood vessels, at a location where a blood flow obstruction has been previously diagnosed.

11. A system for treating a human subject to remove plaque deposits from blood vessels and vasculature of the human subject, comprising:
a plurality of superparamagnetic nanoparticles for introducing into the bloodstream of a human subject;
an electromagnetic field generator for generating a rapidly changing, time-varying electromagnetic field external to the human subject, the electromagnetic field having sufficient magnetic field strength to encompass the human subject, wherein the plurality of superparamagnetic nanoparticles is distributed substantially uniformly though the vasculature of a human subject;
a control system for controlling the electromagnetic field generator for controlling the time-varying electromagnetic field generating an oscillating magnetic effect magnetic to impart randomized vibrational motion to the superparamagnetic nanoparticles throughout the bloodstream of the human subject, wherein the agitating motion of the superparamagnetic nanoparticles vibrate while passing through the vasculature of the human subject, so as to substantially dislodge and dissolve accumulated plaque in the individual blood vessels; and
a unipolar magnetic field, removing the superparamagnetic nanoparticles from the bloodstream of the human subject.

12. The system of claim 11, wherein the control system includes a programmable electronic controller for controlling at least the electromagnetic field strength and operational duration of the electromagnetic field.

13. The system of claim 12, wherein the programmable controller provides selection of superparamagnetic nanoparticles parameters, at least including nanoparticles vibration rate, nanoparticles vibration magnitude, and nanoparticles spin or rotation.

14. The system of claim 12, wherein the programmable controller, through control of the electromagnetic field parameters, can impart a stirring motion to the individual paramagnetic nanoparticles to increase interaction of the nanoparticles with plaque deposits in the human subject's blood vessels.

15. The system of claim 12, wherein the programmable controller, through control of the electromagnetic field parameters, provides nearly uniform distribution of the nanoparticles in the vasculature of the human subject.

16. The system of claim 12, wherein the programmable controller, through control of the electromagnetic field parameters, can concentrate location of the superparamagnetic nanoparticles in one part of the body at the conclusion of the treatment, to provide for less invasive removal of the nanoparticles at one location.

17. The system of claim 12, wherein the programmable controller provides operational control of all phases of the treatment, including uniform distribution of the nanoparticles, vibrational parameters of the nanoparticles, and concentration of the nanoparticles at a removal point in the human subject at the conclusion of the treatment.

18. The system of claim 11, wherein the superparamagnetic nanoparticles have a diameter of less than 500 nm to provide for optimal distribution of the nanoparticles throughout the blood stream of the human subject and are coated with a polymer to minimize damage to the endothelial cells of the blood vessels.

19. The system of claim 11, wherein the superparamagnetic nanoparticles are coated with a therapeutic for dissolving plaque.

20. The system of claim 11, further including a medical imaging system for monitoring location parameters of the superparamagnetic nanoparticles, during treatment.
